(19) United States
(12) Patent Application Publication
(10) Pub. No.: US 2013/0090579 A1
(43) Pub. Date: Apr. 11, 2013

(54) Pulsed Cavitational Therapeutic Ultrasound With Dithering

(71) Applicants: Charles A. Cain, Ann Arbor, MI (US); Tzu-Yin Wang, Taichung City (TW)

(72) Inventors: Charles A. Cain, Ann Arbor, MI (US); Tzu-Yin Wang, Taichung City (TW)

(21) Appl. No.: 13/648,955

(22) Filed: Oct. 10, 2012

Related U.S. Application Data
(60) Provisional application No. 61/545,458, filed on Oct. 10, 2011.

(51) Int. Cl. A61N 7/00

(52) U.S. Cl. CPC A61N 7/00
USPC 601/2

(57) ABSTRACT

Cavitation memory effects occur when remnants of cavitation bubbles (nuclei) persist in the host medium and act as seeds for subsequent events. In pulsed cavitational ultrasound therapy, or histotripsy, this effect may cause cavitation to repeatedly occur at these seeded locations within a target volume, producing inhomogeneous tissue fractionation or requiring an excess number of pulses to completely homogenize the target volume. Cavitation memory can be removed with a dithering technique. The spatial-temporal memory effect of micro-bubbles can be defeated by (1) passive temporal dithering, (2) active dithering, or (3) use of therapy pulses above the de novo threshold.

### CROSS REFERENCE TO RELATED APPLICATIONS

This application claims the benefit under 35 U.S.C. 119 of U.S. Provisional Patent Application No. 61/545,458, filed Oct. 10, 2011, titled “Pulsed Cavitational Therapeutic Ultrasound with Dithering”, which application is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### US GOVERNMENT RIGHTS

This invention was made with government support under NIH R01 CA134579 and R01 EB008998 awarded by the National Institutes of Health. The Government has certain rights in the invention.

### FIELD OF THE DISCLOSURE

The present disclosure generally relates to pulsed cavitational ultrasound treatment of tissue. More specifically, the present disclosure relates to improving methods of performing histotripsy therapy by minimizing or reducing the effects of bubble cloud memory effect.

### BACKGROUND

As described in, e.g., US 2008/0319356 and US 2010/0069797, pulsed cavitational ultrasound can be used to homogenize focal tissue volumes as part of a therapeutic procedure. In such therapies, a cavitation bubble cloud is initiated in the target tissue and an ultrasound therapy sequence is transmitted into the target tissue to interact with the bubble cloud to produce the desired tissue effect, such as homogenization of the tissue.

Histotripsy is a relatively new form of ultrasound cavitation therapy for non-thermal treatment of tissue. Histotripsy depends on consistent initiation and maintenance of energetic bubble clouds in response to many very short high intensity ultrasound pulses. Individual microbubbles—most of which are significantly below 100 micrometers in diameter—act as surgical end effectors to fragment and homogenize target tissue. The bubble clouds, once initiated, can leave behind much smaller remnant microbubbles that can last many milli-seconds. These remnant micronuclei reduce the acoustic intensity threshold for subsequent bubble cloud generation. This ability to initiate a bubble cloud at high intensity and to sustain it at much lower intensities, because of the remnant micro-nuclei memory effect, can be very useful as a strategy to minimize thermal effects that can significantly compromise an overall treatment procedure.

### BRIEF DESCRIPTION OF THE DRAWINGS

FIG. 1a shows one embodiment of a therapeutic histotripsy transducer, and FIG. 1b illustrates a pressure waveform of the therapeutic transducer of FIG. 1a under free-field conditions.

FIG. 2 illustrates an experimental setup comprising a transducer and phantom submerged in a tank.

FIGS. 3a-3d illustrate examples of converting grayscale images to binary images for the cavitation bubble clouds (panel (a) to (b)) and the lesions (panel (c) to (d)).

FIG. 4 shows representative cavitation patterns induced during the treatments with decreasing time intervals between successive pulses.

FIG. 5 plots the cross correlation coefficients between the cavitation patterns produced in successive pulses with varying Δt's.

FIG. 6 shows the overlay of the bubble images during the treatments with decreasing Δt's.

FIG. 7 illustrates the integrated bubble areas during the treatments with varying Δt's.

FIG. 8 shows representative lesion images during the treatment illustrate the lesion development process with decreasing Δt's.

FIG. 9 shows the normalized damaged areas during the treatments with different Δt's.

FIG. 10 shows lesions produced in ex vivo livers using 1000 pulses when the time intervals between pulses was decreased from 200 ms (FIG. 10a) to 2 ms (FIG. 10f).

### SUMMARY

While this bubble cloud memory effect can be quite useful in minimizing the thermal potential of an overall treatment (by, e.g., allowing the therapy session to progress at overall lower average intensities), it can have deleterious effects. The memory effect has not only a temporal component lasting from therapy pulse to pulse, but also a spatial effect. The first initiating pulses, at higher intensity, generate bubble clouds in the focal sub-volume that are not uniformly distributed in bubble density. Moreover, once initiated, the remnant micro-nuclei persist to the next pulse (the temporal component) and in the same location. The bubble densities in the bubble clouds for subsequent pulses have a similar spatial distribution to previous pulses, thereby yielding both a temporal and spatial component of inter-pulse memory.

The “spatial memory” component, however, can have very deleterious consequences. In some situations, the cavitation bubble cloud may have a non-uniform and non-random spatial distribution of bubble densities wherein the bubbles are formed preferentially in one sub-space of the focal volume and are not initiated in adjacent sub-volumes. This effect can persist over many pulses or even over all the pulses of the whole treatment procedure. This memory effect may be caused by surviving remnant micronuclei from the previous pulses.

Whatever the cause, the result can be that some parts of the focal volume are rapidly homogenized while leaving islands of relatively untreated tissue. These untreated islands can be homogenized by the slow process using additional treatment pulses and the expansion outwards in response to each pulse from the homogenized sub-volumes. The process results in some sub-volumes of the focus being over-treated while the more protected zones are inefficiently (and ineffectively) homogenized from the edges outwards of the preferred zones. This uneven distribution of bubble densities and consequent uneven effect on the target tissue significantly increases treatment times. Furthermore, because the focal treatment zone is not treated uniformly over repeated therapy pulses, feedback schemes that have insufficient resolution to see the treated and untreated small sub-volumes are likely to give erroneous results, perhaps registering a volume with intermediate fractionation. In cancer treatment, untreated sub-volumes in a larger treated volume made up of many focal treatment zones could leave viable cells, thereby allowing a tumor to regrow after treatment.

The present invention relates to techniques to make the tissue fragmentation and consequent homogenization progress in a spatially random manner to create a more homogeneous lesion, as averaged over many pulses over the whole focal volume. More specifically, the invention relates to approaches to defeat the memory mechanisms described above, thereby making the bubble clouds randomly distributed in response to each pulse and not dependent on the bubble locations of any previous bubble cloud. This approach helps avoid over-treatment of one tissue sub-volume at the expense of another relatively untreated tissue sub-volume.

The invention significantly decreases the number or pulses necessary to homogenize a given focal volume. Pulse randomization greatly decreases treatment time and assures a more accurate assessment by imaging feedback schemes that quantify the degree of fractionation of the treatment volume. Since some of the focal tissue sub-volumes (treated or untreated) are below the resolution limit of the feedback approach employed, micro-untreated zones may go unnoticed. If the spatial fractionation proceeds randomly, from pulse to pulse, the focal tissue sub-volume will completely and homogeneously be fractionated in far fewer pulses.

There are at least three ways to defeat this spatial-temporal memory effect to obtain a truly random spatial distribution of micro-bubble density within each focal subvolume: (1) Passive temporal dithering; (2) Active dithering; and (3) Use of therapy pulses above the de novo threshold.

Passive Dithering: In this method, one simply waits for the remnant micronuclei to disappear. Over a period of around 1 ms to around 10 ms, the remnant nuclei largely dissolve, allowing the next pulse to generate bubbles in locations largely independent from previous bubble locations within the focal subvolume. This approach works effectively but has the down-side of limiting the PRF (pulse repetition frequency) if spatial-temporal focal scanning is not used. If focal scanning is used, other focal zones of the overall treatment volume (made up of many contiguous focal zones, or focal subvolumes), can be treated while waiting for the previous subvolumes to time-out (in micronuclei memory terms). Thus a treatment can progress rapidly with the transducer being fully utilized temporally if enough focal zones are employed.

Active Dithering: In this approach, a combination of highly localized pulses, with spatially and temporarily modulated parameters designed to spatially redistribute the memory micronuclei, either by active nuclei movement, or by “deleting” the nuclei by greatly reducing or increasing nuclei size beyond the limits of usefulness as seeds for generating bubbles in any subsequent therapy pulses. This approach is also described herein as “focal sharpening.”

Exceeding the De Novo threshold: This approach is perhaps conceptually the least complex but requiring very specific combinations of transducer-driver systems wherein very short (e.g., single cycle) pulses are generated with at least one (or more) of the negative pressure half cycles exceeding the level where all the tissue in the volume spontaneously cavitates. The bubble spatial distributions within the supra-threshold focal sub-volume are relatively randomly distributed spatially. Moreover, because the whole supra-threshold subvolume exceeds the de novo threshold (by definition), pre-existing memory remnant micronuclei are just part of the overall distribution of initiated bubbles but not defining the overall spatial distribution of bubble density within the generated bubble cloud within the focal subvolume. This is a very effective and temporally efficient approach, with minimal potential for undesired thermal consequences, because only single cycle pulses can be used (as long as the negative half cycle exceeds the de novo threshold).

The significance of the above methods and suggested technology is two-fold. Firstly, “dithering,” as described herein, optimally distributes the histotripsy “dose,” in both space and time, so that no volume is overtreated while waiting for full fractionation of another volume. This even distribution of therapy dose resulting from the dithering process results in the most efficient use of time and acoustic energy (and cavitational dose), thus reducing both thermal consequences and treatment time. Treatment times can be critical in treatment of large volumes and could limit effective clinical acceptance (commercially) and overall range of application.

Moreover, dithering results in a randomly distributed progression of tissue fractionation that may be critical in use of imaging feedback to assess, in real time, how the treatment is progressing. It assures that completely fractionated and untreated islands, all existing in a single focal subvolume, do not indicate an erroneous result when these islands are below the image resolution capabilities. In such cases, an intermediate result could be indicated leaving islands of untreated tissue that could be disastrous in cancer therapy.

In some embodiments, a method of performing histotripsy therapy is provided, comprising delivering at least one histotripsy therapy pulse to a volume of human tissue to generate acoustic cavitation in the volume of tissue, defeating a spatial-temporal memory effect generated by the acoustic cavitation with a dithering technique, and applying at least one additional histotripsy therapy pulse to form a homogeneous lesion in the volume of human tissue.

In one embodiment, the delivering at least one histotripsy therapy pulse step comprises delivering at least one ultrasound pulse having a peak negative pressure >10 MPa, a duration <50 μs, and a duty cycle <1%.

In another embodiment, the dithering technique comprises passive temporal dithering. In some embodiments, passive temporal dithering comprises limiting a pulse repetition frequency of the histotripsy therapy pulse. In other embodiments, passive temporal dithering comprises waiting for remnant micronuclei to disappear before initiating another histotripsy therapy pulse. In one embodiment, the waiting step comprises waiting for a period of approximately 1 ms to approximately 10 ms.

In some embodiments, the dithering technique comprises active dithering. In one embodiment, active dithering comprises applying highly localized histotripsy pulses with spatially and temporarily modulated parameters configured to spatially redistribute remnant micronuclei.

In another embodiment, the dithering technique comprises exceeding a De Novo threshold with the histotripsy therapy pulse. In some embodiments, the exceeding step further comprises applying a histotripsy therapy pulse with a negative half cycle exceeding a level where the entire volume of tissue spontaneously cavitates.

### DETAILED DESCRIPTION

Histotripsy therapy pulses can be delivered to create acoustic cavitation induced by high intensity (peak negative pressure >10 MPa) extremely short (<50 μs) ultrasound pulses at low duty cycles (<1%) has been shown to mechanically fractionate soft tissue in well-controlled manner. This process results in soft tissue (‘histo-’) disruption (“-triply”), which has given rise to the term “histotripsy”. Histotripsy has been actively investigated as a tool for non-invasive tissue ablation. Recent studies demonstrated promising results that histotripsy can non-invasively and precisely produce lesions in the target regions in many in vivo models.

For cavitation-based therapy like histotripsy, the initiation and maintenance of the cavitation process is highly affected by the small gas bubbles in a host medium that serve as nuclei for cavitation. These nuclei may preexist in the host medium as gas pockets adhering to crevices on particles or stabilized with organic skin (“stabilized” nuclei), or they may form as fragments of bubbles that persist from collapse of transient cavities (“unstabilized” nuclei). The unstabilized nuclei may become new cavitation sites that sustain subsequent cavitation events. This phenomenon is referred as the cavitation memory effect.

The cavitation memory effect may be advantageous for cavitation-based therapies when the acoustic pressure is insufficient to consistently produce cavitation with each single pulse. In this case, the existence of the cavitation memory, or the persistent nuclei, may help sustain or enhance the cavitation process. This concept has been applied to improve the stone fragmentation efficiency in lithotripsy, or to enhance the tissue fractionation in histotripsy.

Despite the advantage of sustaining the cavitation process, the cavitation memory could be disadvantageous in some cases. One possible disadvantage is that the cavitation bubbles may repeatedly occur at the same locations within a focal volume in response to each pulse due to the presence of the cavitation nuclei. If the spatial distribution of the bubbles is not sufficiently dense, the areas where the cavitation bubbles repeatedly occur are over-treated while the rest areas remain under-treated. This can result in inhomogeneous tissue disruption even within a single focal volume, producing islands of structurally intact tissues in the treatment volume when a small dose is applied. These islands of intact tissues could be detrimental for applications where complete tissue removal is desired (e.g., tumor therapy). In addition, when quantitative tissue characterization methods (e.g., ultrasound spectrum analysis) are used to assess the treatment outcomes, a misleading metric may be produced as an average of fully homogenized and non-homogenized zones.

To completely and homogeneously fractionate the target volume, strategies to break the memory-induced repeated cavitation pattern are needed. One strategy is to overdose the target volume until the cavitation bubbles migrate (sometimes slowly) to different locations in the target volume. However, this may cause inefficient use of energy and prolonged treatment time. Here we propose a dose-efficient strategy to achieve complete and homogeneous tissue fractionation by removing the cavitation memory. The removal of the memory should cause cavitation bubbles to occur at random locations in response to each pulse. As long as the pressure amplitude is high enough so that the cavitation can be consistently induced in each pulse, this random pattern would allow the target volume to be homogeneously fractionated for fewer pulses.

This disclosure investigates the cavitation pattern, i.e., spatial distribution of the cavitation bubbles, in response to each histotripsy pulse and the corresponding lesion development process for different levels of cavitation memory. It is hypothesized that the cavitation memory would decrease with time as the persistent bubbles diffuse, dissolve, and redistribute to new random locations. As such, the level of the persistent memory can be manipulated (passively) by increasing the time interval between successive pulses. Experiments were performed both in the red blood cell (RBC) tissue phantoms and in ex vivo liver tissues. The former allowed for direct visualization of the locations of the cavitation bubbles and the corresponding lesion development process in real time using high speed photography; the latter provided validation of the memory effect in real tissues with histological examinations. This study illustrates a significant effect of cavitation memory on the treatment progression, providing basis for future design of dose-efficient treatments.

### Methods

Sample Preparation

An agarose-based RBC tissue phantom that allows for direct visualization of cavitation and the resulting damage was used to study the impact of the cavitation patterns on the lesion development process. The phantom was prepared with 1% agarose powder (Type-VII, Sigma-Aldrich, St. Louis, Mo.) and 5% v/v RBCs mixed in normal saline. The phantom was constructed such that a thin (˜0.5 mm) RBC-gel layer was suspended between two thick (˜2.5 cm) transparent gel layers. The RBC-gel layer becomes transparent in the locations where the RBCs are damaged by cavitation, likely because the cell content (hemoglobin) is released. The transparent damaged regions are visible within a few milliseconds, and become fully developed within 1 s. This period is likely the time required for sufficient hemoglobin to be released to the medium so that the light can penetrate through the damaged locations. The cavitation bubbles induced in the RBC phantom can be easily detected as dark shadows on backlit optical images. During the treatments, the cavitation bubbles and the lesions can be concurrently recorded, allowing for studying the direct impact of cavitation on the lesion development process.

To validate the cavitation memory effect on treatments in real tissues, experiments were also performed in freshly excised canine livers. The canine livers were obtained from healthy research canines, placed in degassed (20-30% gas saturation) saline at room temperature, and used within 3 hours of harvest. The liver tissues were sectioned into approximately 9 cm×9 cm×6 cm blocks and sealed in plastic bags filled with saline before experimentation.

Ultrasound Generation and Calibration

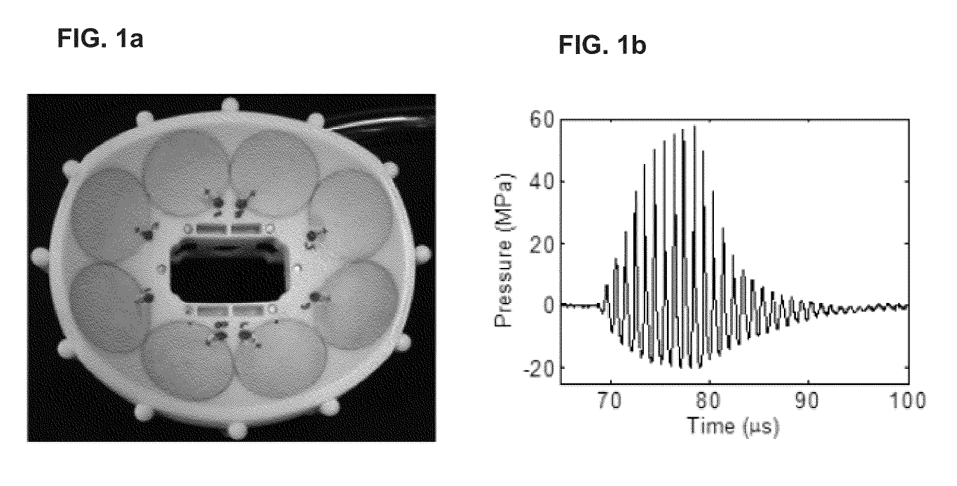

FIG. 1a shows one embodiment of a therapeutic histotripsy transducer, and FIG. 1b illustrates a pressure waveform of the therapeutic transducer of FIG. 1a under free-field conditions. In one experiment, a custom-built 1-MHz F#-0.6 transducer was used to generate therapeutic ultrasound pulses. In one embodiment, the transducer has 8 identical 2-inch diameter PZT disks mounted in an elliptical concaved plastic housing with 18 cm diameter in the long axis, 16 cm diameter in the short axis, and a radius of curvature of 106 mm (FIG. 1a). In the center of the housing is a 7 cm×4 cm rectangular hole for the insertion of imaging probes. The transducer can be driven by electronic input signals generated by a programmed FPGA board, and amplified by a custom-built class D amplifier.

The pressure waveform and beam profiles of the therapeutic ultrasound were obtained in degassed water under free field conditions using a custom-built fiber optic hydrophone with an active element of 100 μm in diameter. Ultrasound pulses of 10 cycles in duration at 1 MHz center frequency were used in all treatments. The peak negative (P−) and peak positive (P+) pressures were 21 and 59 MPa, respectively (FIG. 1b). The −6-dB beamwidths were estimated on both P− and P+ pressure profiles at P−/P+ pressure of 18/48 MPa. The −6-dB beamwidths measured 1.2 mm along the long lateral axis, 1.3 mm along the short lateral axis, and 6.9 mm along the axial direction on the P− pressure profile. For the P+ profile, the −6-dB beamwidths measured 1.0 mm along the long lateral axis, 1.2 mm along the short lateral axis, and 4.8 mm along the axial direction. The beam profiles at the experimental pressure level could not be measured because of the interference from the bubble cloud at the fiber tip. In the experimental setting, the acoustic intensity could be attenuated during the propagation path in the tissues. Given the 0.5 dB/cm/MHz attenuation in the liver, and a 1 cm mean propagation path, the P− pressure was likely 20 MPa. The P+ pressure was likely decayed more significantly to <56 MPa due to nonlinear attenuation.

Ultrasound Treatment Parameters

The treatments were performed using various time intervals (Δt) between successive pulses, with fixed doses of 500 pulses for the RBC phantoms and 1000 pulses for the ex vivo tissues. The intervals, Δt, varied from 2, 10, 20, 50, 100, to 200 ms in different treatments, as our previous study showed that the cavitation nuclei can persist up to several tens of ms after a histotripsy pulse. These intervals were equal to or longer than those commonly used in the histotripsy treatments such that minimal thermal effects would occur in this study. A total of 65 treatments were performed on the RBC phantoms, resulting in a sample size of 10-12 for each Δt. A total of 26 ex vivo treatments were performed on the livers, resulting in a sample size of 4-5 for each.

Ultrasound Treatment and Monitoring

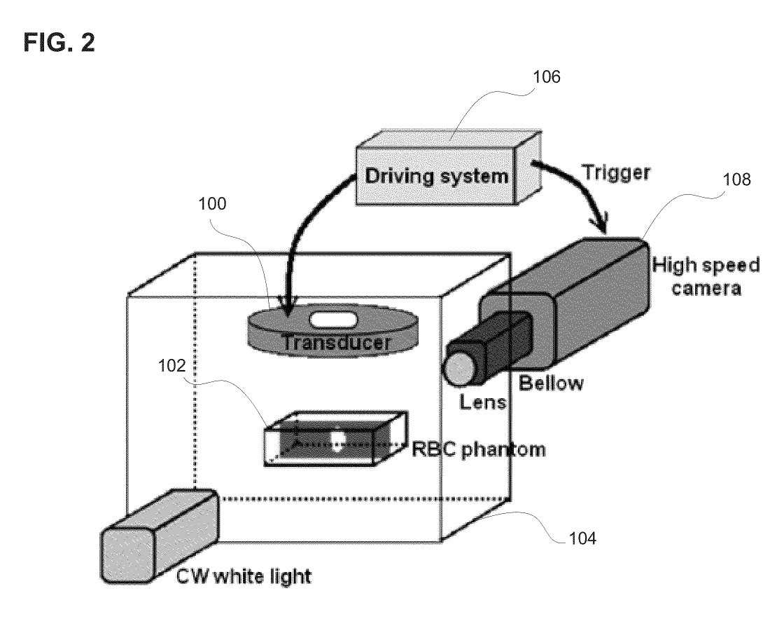

FIG. 2 illustrates an experimental setup comprising a transducer 100 and phantom 102 submerged in a tank 104. The transducer can be driven by driving system 106. An imaging system 108 (e.g., a high-speed camera) can be mounted on or near the transducer, optionally perpendicular to the ultrasound beam of the transducer such that a projection of the bubble cloud and lesions formed by the transducer can be recorded. Before the treatment, the focus of the transducer was aligned with the RBC layer in the phantom using the following approach. A bubble cloud was first generated in the water using brief excitation of the transducer. The location of the bubble cloud was indicated using two 1-mm-wide 5-mW laser beams, one along and the other perpendicular to the ultrasound beam, crossed at the middle of the bubble cloud. The phantom was then placed in the tank such that the RBC layer was aligned with the laser beams and thus to the ultrasound beam and along the long axis of the transducer (FIG. 2). For the ex vivo treatments, the tissue samples were positioned in a similar fashion.

During the treatment, the cavitation bubble clouds and the lesions were imaged with a 12 bit, 1280×960 pixel high speed photography. Backlighting was provided using a 300 W continuous white light lamp. This lighting allowed for a short exposure of 2 μs for each frame. The frame size was adjusted to be 16 mm×12 mm using a Tominon macro-bellows lens attached to the camera. This image size ensured imaging of the overall bubble cloud and the entire lesion.

The imaging was performed after each pulse throughout the entire treatment. The bubbles were imaged 10 μs after the pulse because the spatial extent of the bubble cloud at this time corresponds well with that of the lesion. The lesion image was taken at mid-period after the pulse when the cavitation bubbles disappeared on the optical images. This timing allowed for imaging of the lesions without interference from the bubbles for all Δt's used in this study. Because the lesions may take several milliseconds and up to 1 s to become fully developed, another lesion image was taken approximately 5 seconds after the entire treatments to ensure that the maximum extent of the damage was imaged.

Cavitation Pattern Analysis

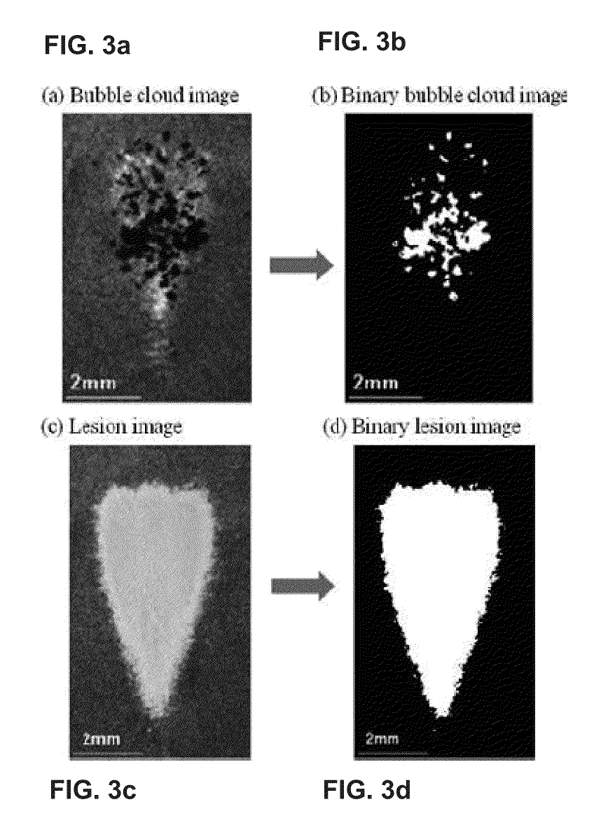

FIGS. 3a-3d illustrate examples of converting grayscale images to binary images for the cavitation bubble clouds (panel (a) to (b)) and the lesions (panel (c) to (d)). The cavitation bubbles appeared as dark shadows on the backlit images (e.g., FIG. 3a), which can be easily distinguished from the background by the brightness. To detect the cavitation bubbles on the images, a pixel brightness threshold was set at the mean−5×standard deviation of the pixel brightness in a 2 mm×2 mm region in the intact background area (light gray area on the images). The pixels with intensities lower than this threshold were considered in the areas of the cavitation bubbles. Using this threshold, the grayscale image was converted to a binary bubble image where 1 (white) represented the presence of bubbles and 0 (black) represented the absence of bubbles (FIG. 3b).

The effects of varying intervals between successive pulses on the cavitation patterns were studied by measuring: 1) the similarity, i.e. cross correlation coefficient, between cavitation patterns in successive pulses, and 2) the integrated bubble area as the pulse number accumulated. The former assessed the level of the persistent cavitation memory. The latter indicated how fast the target volume can be completely “exposed” to or treated with the bubbles. The cross correlation coefficient between cavitation patterns was calculated using the following equation:

$$Cross correlation coefficient = \frac{{Σ}_{i} {X}_{k} ( i ) {X}_{k + 1} ( i )}{\sqrt{{Σ}_{i} {{X}_{k} ( i )}^{2}} \sqrt{{Σ}_{i} {{X}_{k + 1} ( i )}^{2}}}$$

where Xₖ(i) and Xₖ₊₁(i) are the binary bubble images in the k-th and (k+1)-th pulses, and i is the pixel index on the images. This coefficient was computed for each pair of successive pulses through the entire treatment, i.e., 1≦k<500. To measure the integrated bubble area, an overlay of the bubble images was first formed for the k-th pulse by overlaying the binary bubble images from the first to the k-th pulse. The overlay image was also expressed in a binary format, where 1 indicated the presence of bubbles and 0 indicated the absence of bubbles in any pulse between the first and the k-th pulses. The integrated bubble area was computed by summing the areas with the presence of the bubbles on the overlay of the bubble images. The overlay outlined a region that could potentially be damaged in the treatments. The increasing trend of the integrated bubble area as the pulse number accumulated may predict the lesion developing trend.

Lesion Analysis

The damaged areas were significantly brighter than the intact areas (e.g., FIG. 3c), and could be detected using a similar threshold approach. A pixel brightness threshold was set at the mean+5×standard deviation of the pixel brightness in a 2 mm×2 mm region in the intact background area. Pixels with brightness higher than this threshold were considered “damaged.” Using this threshold, the grayscale images were converted to binary lesion images where 1 (white) represented “damaged area” and 0 (black) represented “intact area” (FIG. 3d).

To study the lesion development process, the following analysis procedures were performed. First, the treatment zone was selected on the post-treatment lesion image by outlining a region which encompassed the maximal extent of the lesion, and shaped like the overlay bubble image obtained in the previous section (cavitation pattern analysis). Next, the damaged area was calculated for each lesion image captured during the treatment by integrating the areas identified as “damaged” in the treatment zone. The damaged area was further normalized to the area of the treatment zone, resulting in the normalized damaged area. This normalized damaged area was compared for treatments using different time intervals between pulses.

Histological Examination

Histological examination was conducted for the lesions produced in ex vivo tissues. After the treatments, the tissues were fixed in formalin and prepared for hematoxylin and eosin (H&E) sections. The lesions were sectioned longitudinally along the ultrasound beam. Multiple 4-μm thick H&E sections were made through the lesions with a 1-mm step size. The sections with the maximum spatial extent of damage both laterally and axially were examined.

### Results

Cavitation Patterns

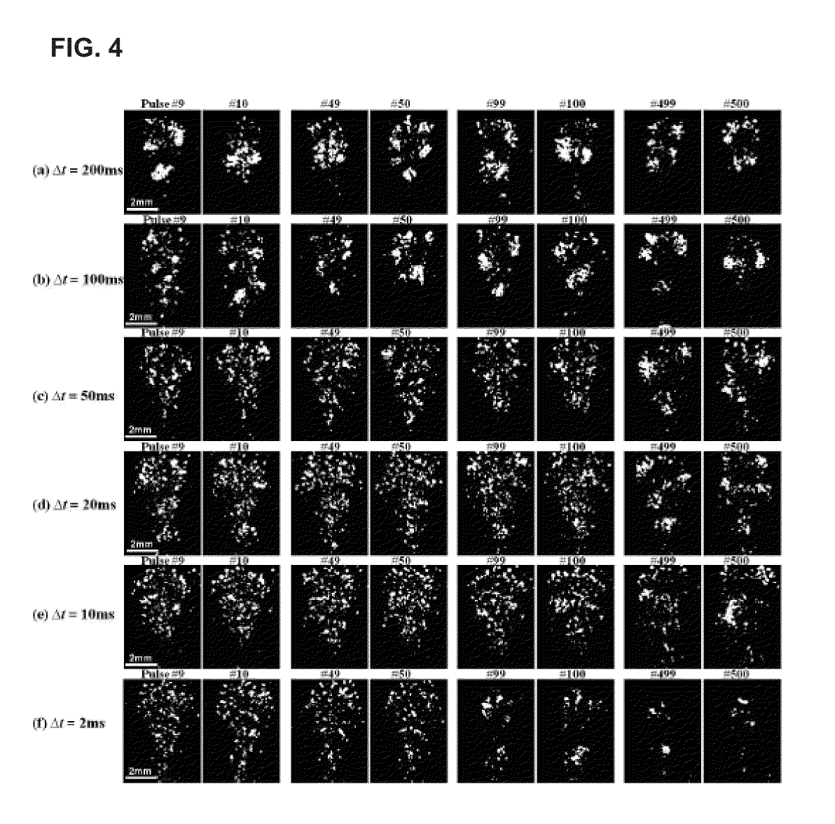

Representative cavitation patterns induced during the treatments with decreasing time intervals between successive pulses are shown in FIG. 4. Cavitation patterns generated by successive pulses are shown during the treatments when the time interval between pulses is decreased. The cavitation patterns in successive pulses appeared distinctly different in response to each pulse when the time interval between pulses was longer. The locations of the cavitation bubbles in response to each pulse were highly dependent on the time interval. When Δt was ≧100 ms, the cavitation bubbles were induced at distinctly different locations in response to each pulse. s Δt was decreased to less than 100 ms, many bubbles appeared at the same locations in each pulse. This repeated pattern was most prominent at the beginning of the treatment, and became less significant as the pulse number increased.

The spatial extent of the bubble cloud was also affected by Δt. The bubble cloud appeared in a more confined area when Δt was ≧100 ms, and expanded as Δt decreased from 100 to 10 ms. s Δt further decreased to 2 ms, the bubble cloud was as large as that produced with Δt ranging between 100-10 ms at the beginning of the treatment, but appeared in a much smaller area around the center of the focus as the pulse number increased.

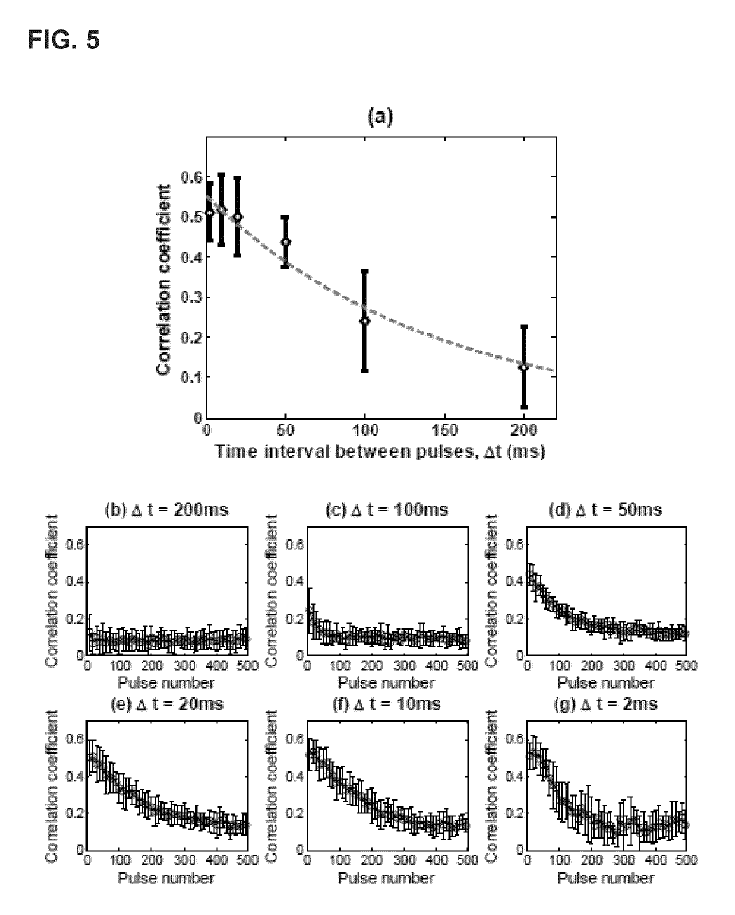

The cross correlation coefficients between the cavitation patterns produced in successive pulses with varying Δt's are plotted in FIG. 5. The cross correlation coefficient decreased exponentially with increasing Δt's. This exponential decrease was particularly significant at the beginning of the treatment (i.e., within 100 pulses). For example, the correlation coefficient at the 10th pulses during the treatments with varying Δt's deceased from 0.5±0.1 to 0.1±0.1 as Δt increased from 2 to 200 ms. These data were well fitted by an exponential curve (R²=0.96, FIG. 5a). The exponential decay in the correlation coefficient became less significant as the treatment continued because the correlation coefficient might change with increasing numbers of pulses (FIG. 5b-f). At the longest Δt (i.e., 200 ms), the cross correlation coefficient remained low at 0.1±0.1 throughout the entire treatment. When Δt decreased to ≦20 ms, the correlation coefficient decreased from ˜0.5 to ˜0.1 as the pulse number increased from 0 to 500.

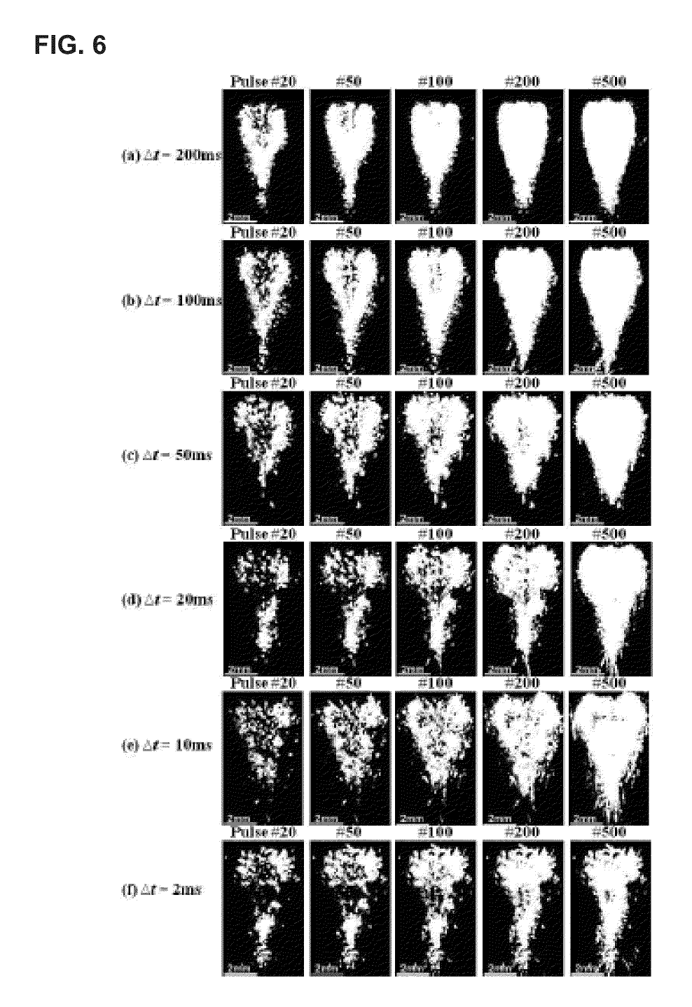

The overlay of the bubble images during the treatments with decreasing Δt's are shown in FIG. 6. When Δt was long, the bubbles occurred at random locations in each pulse. Therefore, the focal volume was fully exposed to the cavitation bubbles within a small number of pulses. When Δt was decreased, the focal volume was not fully exposed to the cavitation bubbles until a larger number of pulses were delivered. For instance, the focal volume was fully exposed to the bubbles within 100 pulses at Δt=200 ms (FIG. 6a); however, it was not fully exposed until 500 pulses were delivered when Δt was decreased to 50 ms (FIG. 6c). When Δt was further decreased, some regions in the focal volume were never exposed to the cavitation bubbles during the entire treatment (FIG. 6e, f).

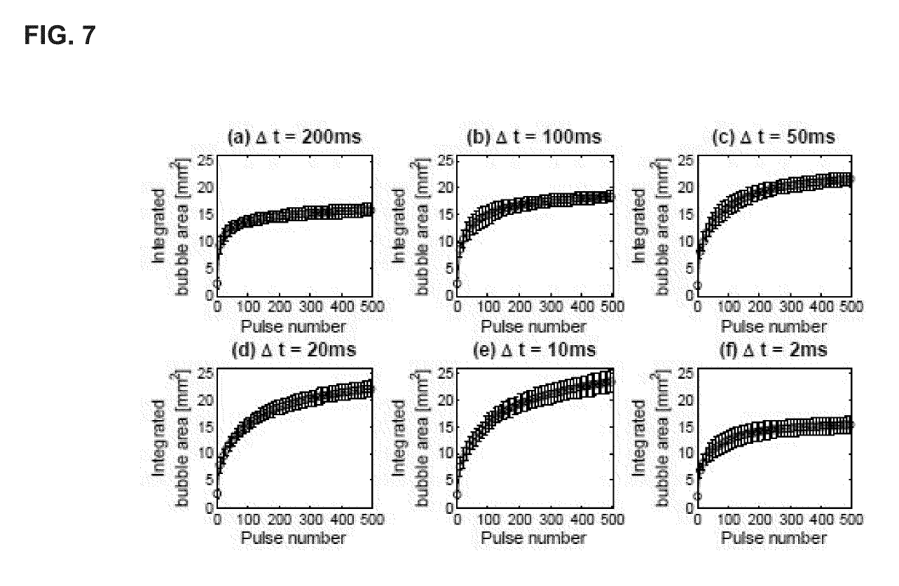

The integrated bubble areas during the treatments with varying Δt's are shown in FIG. 7. When Δt was ≧100 ms, the integrated bubble area rapidly increased with each additional pulse at the beginning of the treatment, and reached a plateau at 100 pulses (FIGS. 7a and b). This trend indicated that the target volume was fully exposed to the cavitation bubbles within a small number of pulses. As Δt was decreased from 100 to 10 ms, the increase in the integrated bubble areas slowed down, and a plateau was never observed within 500 pulses (FIGS. 7c and e). When Δt was decreased to 2 ms, a plateau in the integrated bubble area was observed again (FIG. 7f). This plateau occurred for a different reason: the spatial extent of the bubble cloud decreased as the pulse number increased, thus limiting the growth of the overall bubble coverage area. This behavior is evidenced in FIG. 4f as well where the spatial extent of the bubble cloud is limited in later pulses.

Lesion Development Process

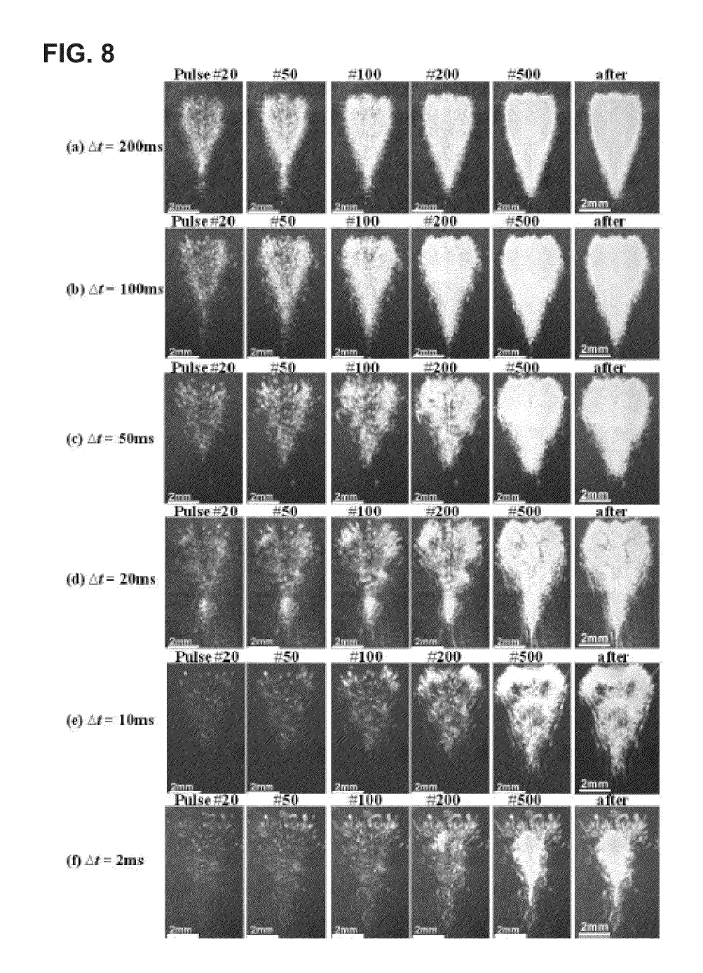

Representative lesion images during the treatment illustrate the lesion development process with decreasing Δt's (FIG. 8). In all treatments, damaged areas were detected after each pulse. The damaged area increased with increasing pulse numbers. The lesion developed more rapidly with each pulse for longer Δt's. Furthermore, the lesion appeared to be homogeneously treated, and possessed a smooth and well-defined boundary with no or very few residual intact areas in the treatment zone. As Δt was decreased, the lesions presented a ragged boundary with many residual intact areas.

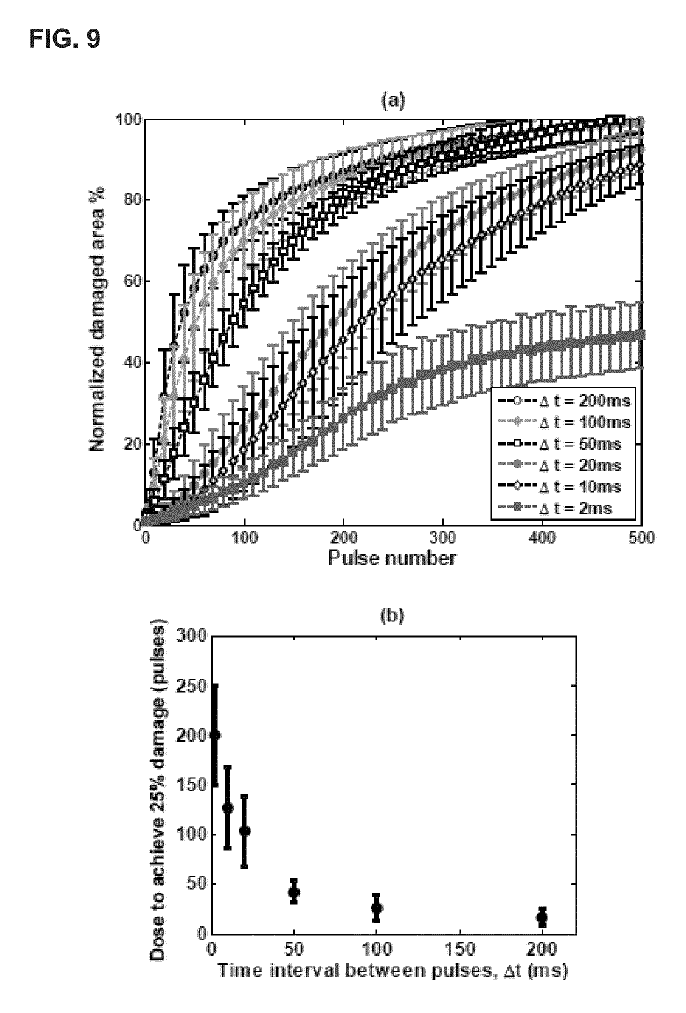

The normalized damaged areas during the treatments with different Δt's are shown in FIG. 9. The normalized damaged area increased more rapidly with each pulse when Δt was ≧100 ms. This increase slowed down as Δt decreased from 100 to 10 ms. The slowest increase was found at the shortest Δt, i.e., 2 ms. After the treatments, complete fractionation of the treatment zone (i.e., 100% damage) was achieved only when Δt was ≧50 ms. The normalized damaged area decreased when Δt was decreased. At Δt=2 ms, only 50% of the treatment zone was damaged after the treatment. To compare the treatment efficiency for different Δt's, the dose required to achieve 25% damage was calculated (FIG. 9b). As Δt increased from 2 to 200 ms, the dose required for 25% damage decreased from 199±50 to 17±9 pulses, approximately a 12-fold difference. This indicated that the treatment efficiency (defined as damage per pulse) was significantly higher for long Δt's.

Ex Vivo Study

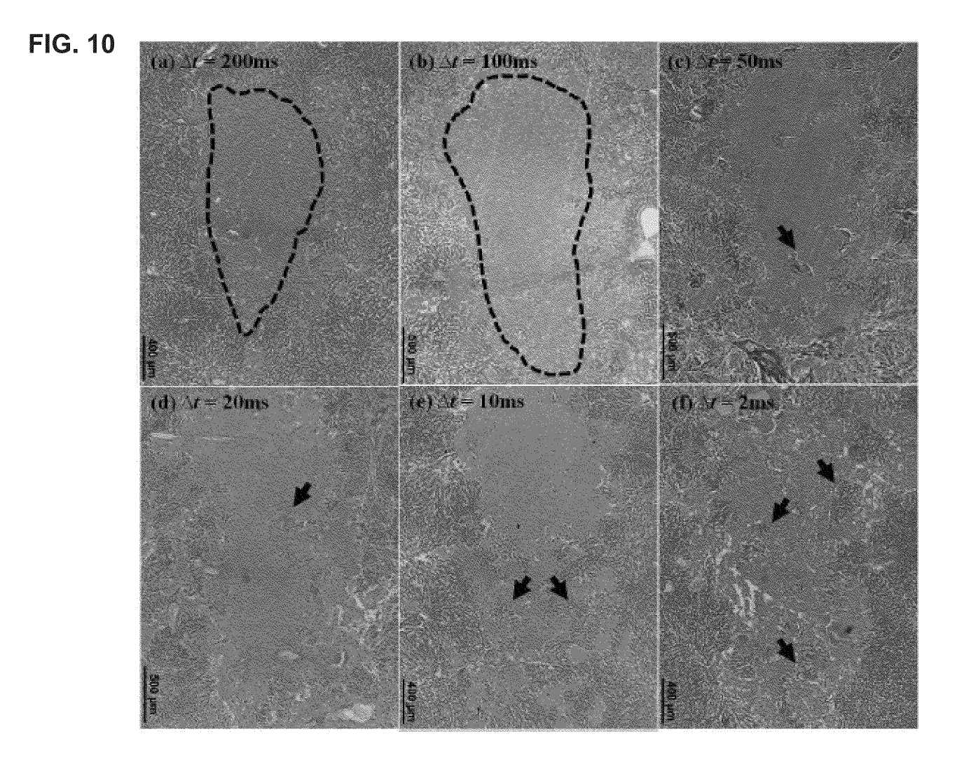

The ex vivo treatment results confirmed that the lesion morphology was highly dependent on the time intervals between pulses. With 1000 pulses applied, when Δt was ≧100 ms, the lesions appeared to be homogeneously and completely disrupted with no or very few recognizable tissue structures in the treatment zone (FIGS. 10a and b). As Δt decreased to 50−20 ms, islands of incompletely disrupted structures were present in the midst of the mostly treated zone (FIGS. 10c and d). As Δt further decreased to below 20 ms, a significant amount of structurally intact tissues remained in the treatment zone (FIGS. 10e and f). These results demonstrated that when the dose was held small, complete tissue disruption was more likely achieved when Δt was increased.

### Discussion

We hypothesized that the cavitation memory may be removed by applying the subsequent pulse after a sufficiently long time interval following the previous pulse and that the removal of the memory may lead to complete and homogeneous tissue fractionation with fewer pulses. These hypotheses were supported by the results. At short time intervals between pulses, the highly correlated cavitation patterns indicated the presence of the cavitation memory. When the time interval between pulses was increased, the memory disappeared. As a result, the cavitation bubbles occurred in random locations in response to each pulse. The random patterns allowed the target volume to be fully exposed to cavitation within a significantly smaller number of pulses, leading to complete and homogeneous tissue fractionation with dramatically fewer pulses.

Despite the benefits of treatments with increased time interval between pulses, the total treatment time can be long if a large volume of tissue is treated by one single focal spot at a time. Since histotripsy pulses are only a few microseconds long, we propose using a 2-D phased-array to steer the focus electronically to other locations within the treatment volume during the time between pulses (˜100 ms). As such, the entire volume can be completely fractionated using the same time that is needed to fractionate a single focal spot. In this way, the treatment time to ablate a large tissue volume may be significantly reduced by increasing the duty cycle of the phased array transducer.

To remove the cavitation memory, this study used a passive approach by increasing the time interval between pulses. In addition to this passive approach, active approaches can be used. For example, persistent bubbles in the periphery of the focus can be actively removed by a nuclei preconditioning pulse delivered before each therapy pulse. A similar pulsing sequence can remove the persistent nuclei in the treatment volume. This pulsing sequence, can use a special pulse to remove the cavitation memory immediately after each pulse. As soon as the cavitation memory is removed, the next therapy pulse can be delivered. Therefore, the time interval between successive pulses for the memory effect to disappear may be substantially reduced.

Although the presence of the cavitation memory caused highly correlated cavitation patterns at the beginning of the treatment, this correlation gradually decreased as the treatment continued. This decreasing correlation likely occurred because the progressive fractionation of the treatment volume, which eventually turned the treatment tissue volume to liquefied homogenate, provided increased mobility for the persistent cavitation nuclei. The similarity between cavitation patterns was therefore decreased. Since this change only occurred in the later stage of the treatment, the overall treatment efficiency remained low compared to that of the treatments with uncorrelated cavitation patterns during the entire treatment.

The decreasing trend in the correlation coefficient of the cavitation patterns with increasing Δt's indicated that the memory effects decayed exponentially with time and disappeared in several tens to hundreds of ms. The decay trend and period corresponded well with those of the residual bubbles that persisted in the treatment volume after a histotripsy pulse. The decay period also corresponded well with the dissolution time of micron size gas bubbles. These suggested that the persistent gas bubbles are an important source causing the memory effect. It is not excluded that the fragments of fractionated tissues or tissue phantoms may also serve as potential cavitation nuclei that contribute to the memory effect.

The normalized damaged area measured during the treatments (FIG. 9a) may be slightly underestimated due to the limitation in the temporal response of the RBC phantoms. The damage was imaged 1-100 ms after each pulse; however, it may take several milliseconds for the lesions to become fully developed. This latency could have caused different amount of underestimation for various Δt's. To evaluate the amount of underestimation, the damaged areas measured at the last pulse of the treatments and 5 s after the treatments (two rightmost columns in FIG. 8) were compared. The difference between the two measurements were found to be 0.1-1.2 mm2, with the maximum occurring for Δt=2 ms. In the worst case, this difference would cause an underestimation of the normalized damaged area by 7%. This amount is small compared to the difference caused by different experimental conditions, and thus should have minimal influence on the trends observed in FIG. 9.

An interesting evolutionary trend of the bubble cloud induced at the shortest time interval between pulses (i.e., 2 ms), or the highest pulsing rate, was observed. The spatial extent of the bubble cloud decreased to a smaller area around the focal center as the pulse number increased. This phenomenon may have resulted from significant decrease in the available cavitation nuclei in the treatment volume. The available cavitation nuclei in the target volume could have been quickly depleted after repetitive pulsing at a high pulsing rate. In addition, the short interval between pulses could have impeded the replenishment of new cavitation nuclei from the surrounding area. The depletion of the available nuclei may have raised the cavitation threshold in the treatment volume, therefore restricting bubbles in a smaller area where the pressure amplitude remained above the new threshold. Further investigation is needed to distinguish the mechanisms behind this phenomenon.

### CONCLUSIONS

This study demonstrated that cavitation memory may have distinct influence on the lesion development process in histotripsy. The cavitation memory effect resulted in highly correlated cavitation patterns, leading to slow development of lesions with each pulse. The removal of the memory effect caused cavitation bubbles to occur in new random locations in response to each pulse, resulting in complete and homogeneous tissue disruption with significantly fewer pulses, i.e., more dose-efficient treatments. Moreover, in real-time monitoring of lesion development, homogeneously disrupted lesions should result in tissue characterization metrics representative of the whole lesion instead of a misleading average of fully homogenized and non-homogenized zones. This may be potentially important in image-guided cancer therapy.

What is claimed is:

1. A method of performing histotripsy therapy, comprising;
delivering at least one histotripsy therapy pulse to a volume of human tissue to generate acoustic cavitation in the volume of tissue;
defeating a spatial-temporal memory effect generated by the acoustic cavitation with a dithering technique; and
applying at least one additional histotripsy therapy pulse to form a homogeneous lesion in the volume of human tissue.

2. The method of claim 1 wherein the delivering at least one histotripsy therapy pulse step comprises delivering at least one ultrasound pulse having a peak negative pressure >10 MPa, a duration <50 μs, and a duty cycle <1%.

3. The method of claim 1 wherein the dithering technique comprises passive temporal dithering.

4. The method of claim 3 wherein passive temporal dithering comprises limiting a pulse repetition frequency of the histotripsy therapy pulse.

5. The method of claim 3 wherein passive temporal dithering comprises waiting for remnant micronuclei to disappear before initiating another histotripsy therapy pulse.

6. The method of claim 5 wherein the waiting step comprises waiting for a period of approximately 1 ms to approximately 10 ms.

7. The method of claim 1 wherein the dithering technique comprises active dithering.

8. The method of claim 7 wherein active dithering comprises applying highly localized histotripsy pulses with spatially and temporarily modulated parameters configured to spatially redistribute remnant micronuclei.

9. The method of claim 1 wherein the dithering technique comprises exceeding a De Novo threshold with the histotripsy therapy pulse.

10. The method of claim 1 wherein the exceeding step further comprises applying a histotripsy therapy pulse with a negative half cycle exceeding a level where the entire volume of tissue spontaneously cavitates.
